(19) United States
(12) Patent Application Publication
(10) Pub. No.: US 2014/0000625 A1
(43) Pub. Date: Jan. 2, 2014

(54) Trach Secretion Catcher

(71) Applicant: Brenda J. Williams, JR., Riverdale, IL (US)

(72) Inventor: Brenda J. Williams, JR., Riverdale, IL (US)

(21) Appl. No.: 13/914,613

(22) Filed: Jun. 10, 2013

Related U.S. Application Data
(60) Provisional application No. 61/689,579, filed on Jun. 8, 2012.

(51) Int. Cl. A61M 16/04

(52) U.S. Cl. CPC A61M 16/047
USPC 128/207.17

(57) ABSTRACT

The invention provides an assistive device specially designed for use in patient trachea care after a tracheotomy surgery providing an easier and more effective means of managing secretions for both a caregiver and a patient. The Trach Secretion Catcher is a wearable pouch that catches excess mucous and seeping liquids from a patient's tracheotomy open area, facilitating quick and easy maintenance while ensuring patient comfort in the process.

### CLAIM OF PRIORITY

This patent application claims priority under 35 USC 119 (e) (1) from U.S. Provisional Patent Application Ser. No. 61/689,579 filed Jun. 8, 2012, of common inventorship herewith entitled, “Trach Secretion Catcher.”

### FIELD OF THE INVENTION

The present invention pertains to the field of surgery recovery, and more specifically to the field of tracheotomy surgery recovery.

### BACKGROUND OF THE INVENTION

The prior art has put forth several designs for tracheotomy surgery recovery aids. Among these are:

U.S. Pat. No. 5,058,579 to Deborah A. Terry and John T. Terry describes a tracheotomy dressing comprising an occlusive adhesive backed dressing, a means for securing a tracheotomy tube to the occlusive dressing and a means for attaching a absorbent pad for collecting tracheal secretions to the occlusive dressing. The tracheotomy dressing is both tieless and gauzeless and provides advantages of a functional as well as a cosmetic nature. U.S. Pat. No. 5,918,599 to Barry F. Shesol describes a tracheotomy tube dressing and support device unit used for holding a tracheotomy tube in place and holding a primary wound dressing, such as a sterile gauze pad, next to a tube insertion site in the front of a patient's neck. The unit includes an elastic bidirectional wrap stretchable in opposite directions along a length of the wrap. One end of the wrap includes hook fasteners for engaging an opposite end of the wrap and holding it in place around the neck. The wrap includes a window opening with a pair of hook fastener strips attached to opposite sides of the window opening and on an outside of the wrap. The hook fastener strips are used for releasable receipt through flange openings in opposite sides of a tracheotomy tube flange. When the hook fastener strips are threaded through the flange openings, the strips are secured to loop fastener landing pads thus holding the tracheotomy tube firmly in place.

U.S. Pat. No. 7,093,598 Jeanne Hanneman and Marie Conn describes a device and a method of use that allows a medical professional to change a patient's tracheotomy dressing in an efficient manner that cuts down both the professional's and patient's discomfort. In the preferred embodiment of the invention, the device has a body that includes a handle, a shoulder and two prongs, however, the handle is optional. The entire lower surface of the body has a concave curve in one embodiment. The handle extends and ends at the shoulder. On the end of the shoulder, extending away from the shoulder, and curving in an upward direction relative to the shoulder, are two prongs. The prongs are parallel to one another and form a U shape, the distance of which is wider than the distance of the diameter of a trach tube. The prongs are designed to fit within the trach drainage sponge. This allows the user of the device to insert the sponge in a simple, smooth manner.

None of these prior art references describe the present invention.

### SUMMARY OF THE INVENTION

It is an object of the present invention to provide an assistive device for managing secretions during tracheotomy care.

### BRIEF DESCRIPTION OF THE DRAWINGS

FIG. 1 is a prototypical back view showing a split gauzer for absorption, a gauzer bottom for secretion absorption and an adjustable hook and loop strap.

FIG. 2 is a functional prototypical front view showing a pouch around a person's neck and an opening in between front and back.

FIG. 3 is an illustrative front view showing straps attached to back of pouch, pieces of hook and loop attachment means attaching back strap to front of pouch, and a covering for tracheotomy open area.

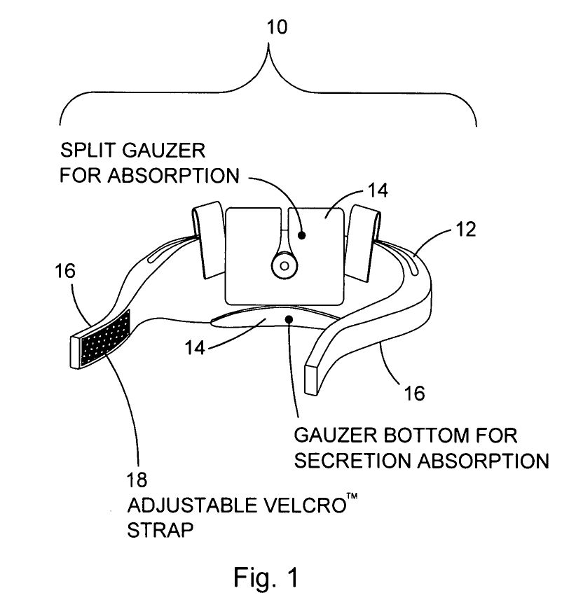

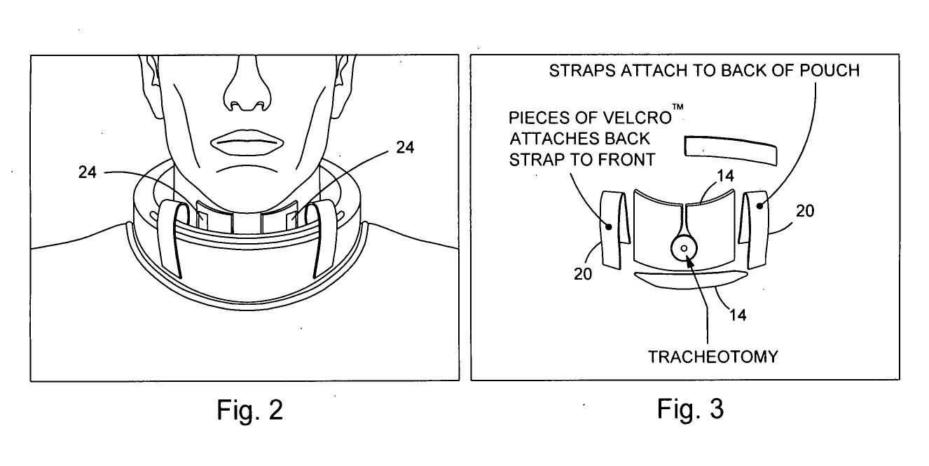

### DETAILED DESCRIPTION OF THE INVENTION

According to an Encyclopedia of Surgery, a tracheotomy is a surgical procedure in which a cut or opening is made in a patient's windpipe or trachea. This surgical procedure sometimes is called a tracheostomy, pharyngotomy or laryngotomy. A tracheotomy or tracheostomy tube, commonly called a trach, is inserted into the opening to bypass an obstruction. This procedure is performed if enough air is not getting to one's lungs, if a person is unable to breathe without help or is having problems with mucus and other secretions getting into the windpipe because of difficulty swallowing. There are several reasons why air is prevented from entering one's lungs. The windpipe is blocked by swelling, a large foreign object, paralysis of the throat muscles, or a tumor or a severe injury to neck, nose, or mouth. The patient is in a coma or needs a ventilator to pump air into the lungs for a long period of time. Tracheotomies commonly are performed on infants who are born prematurely since their lungs are undeveloped at time of birth. Since a trach remains in place for an indeterminate amount of time, daily care of the trach site is required to prevent infection and skin breakdown under a tracheotomy tube and ties. Secretions are monitored and thoroughly removed so airways are unobstructed. Trach care is performed at least once a day and more often if needed. Children with new trachs and those patients on ventilators need trach care several times in a twenty four hour period, as excess secretions are likely to occur. Trach maintenance is a challenging endeavor for a caregiver and a traumatic experience for a patient.

The present invention, hereinafter referred to as the Trach Secretion Catcher, is an assistive device specially designed for use in patient trachea care after a tracheotomy surgery. The invention provides an easier and more effective means of managing secretions for both a caregiver and a patient. The Trach Secretion Catcher is a wearable pouch 10 that catches excess mucous and seeping liquids from a patient's tracheotomy open area, facilitating quick and easy maintenance while ensuring patient comfort in the process. Essentially rectangular in shape, the pouch component 12 of this product measures approximately four to six inches in width. Backed with a plastic material, the pouch 12 contains medical grade gauze 14 for optimal absorption. One convenient size of gauze useful for this purpose is four by four inch square. To facilitate wear for a patient, each side of the Trach Secretion Catcher contains an elongated extension strap 16 that fits around the neck and measures approximately twelve inches in length. With a paper overlay, these wraps contain strips of the hook and loop fastening means 18, commonly known as Velcro™, for security. The pouch also employs hook and loop fastening means 20 to reinforce a connection to the neck wraps. The Trach Secretion Catcher is available in a wide variety of sizes to accommodate any tracheotomy patient including premature babies, infants, children and adults.

In an alternative embodiment, a humidity vent 24 can be incorporated onto the front of the pouch to humidify the intake air. These humidity vents comprise absorbent material such as paper that is saturated with water to allow humidity to enter into the intake air.

Relatively simple in design yet effective in application, a caregiver positions a Trach Secretion Catcher around a patient's neck, placing the pouch at the open area of the tracheotomy while affixing the wraps around the back of the neck. If seeping occurs or excess secretions emit due to patient coughing, the pouch captures the matter, keeping it from transferring to surrounding surfaces. Using the present invention keeps secretions from building up and creating an unsanitary mess when a caregiver is absent. The Trach Secretion Catcher is simply removed from the patient, washed or properly disposed and replaced with a new unit as needed. A Comfortable pouch designed specifically for a trach patient, the present invention ensures that secretions from a patient's tracheotomy area are captured and absorbed as soon as they occur. Both patient and caregiver no longer worry about occlusion or obstruction from secretion buildup. The Trach Secretion Catcher eliminates unsanitary accumulation of discharge on patient clothing, bedding and caregivers. Employing the Trach Secretion Catcher, caregivers expediently complete a trach discharge maintenance process in a mere matter of seconds. With a safe hypoallergenic construction that is conveniently disposable, the Trach Secretion Catcher is a cost effective accessory for patient care for as long as a trach is required.

The Trach Secretion Catcher is an innovative product invention which conceivably revolutionizes tracheotomy care. Simple to operate yet extremely effective in usage, the present invention is an invaluable addition to the medical devices field. Affordably priced, the Trach Secretion Catcher is well received by patients who have undergone tracheotomy surgery as well as those who care for these patients.

Although this invention has been described with respect to specific embodiments, it is not intended to be limited thereto and various modifications which will become apparent to the person of ordinary skill in the art are intended to fall within the spirit and scope of the invention as described herein taken in conjunction with the accompanying drawings and the appended claims.

1. An assistive device for managing secretions during tracheotomy care, comprising a wearable pouch that catches excess mucous and seeping liquids from a patient's tracheotomy open area, facilitating quick and easy maintenance while ensuring patient comfort in the process, wherein the pouch is essentially rectangular in shape, having elongated side straps to fit around the neck of the wearer.

2. The assistive device of claim 1 for managing secretions during tracheotomy care wherein the elongated side straps comprise hook and loop fastening means to adjust the length of the device.

3. The assistive device of claim 1 for managing secretions during tracheotomy care wherein the overall length measures approximately twelve inches in length, and wherein the side straps comprise hook and loop fastening means to adjust the length of the device.

4. The assistive device of claim 3 for managing secretions during tracheotomy care wherein the pouch employs hook and loop fastening means to reinforce connection to the side straps.

5. The assistive device of claim 1 for managing secretions during tracheotomy care available in a wide variety of sizes to accommodate any tracheotomy patient including premature babies, infants, children and adults.

6. The assistive device of claim 1 for managing secretions during tracheotomy care wherein the pouch component of this product measures approximately four to six inches in width, and comprises an opening for receiving the opening of the trach.

7. The assistive device of claim 6 for managing secretions during tracheotomy care wherein the pouch is backed with a plastic material, and wherein the pouch contains medical grade gauze for optimal absorption.

8. The assistive device of claim 7 for managing secretions during tracheotomy care wherein the medical grade gauze is four inch by four inch square.

9. The assistive device of claim 4 for managing secretions during tracheotomy care further comprising means for allowing the user to speak while wearing the device.
